(19) United States
(12) Patent Application Publication
(10) Pub. No.: US 2026/0114820 A1
(43) Pub. Date: Apr. 30, 2026

(54) NUCLEAR MEDICINE DIAGNOSTIC APPARATUS

(71) Applicant: CANON MEDICAL SYSTEMS CORPORATION, Otawara-shi (JP)

(72) Inventor: Motohiro INOUE, Otawara (JP)

(73) Assignee: CANON MEDICAL SYSTEMS CORPORATION, Otawara-shi (JP)

(21) Appl. No.: 19/326,215

(22) Filed: Sep. 11, 2025

(30) Foreign Application Priority Data
Oct. 29, 2024 (JP) 2024-189690

(51) Int. Cl. A61B 5/00; G01N 23/046

(52) U.S. Cl. CPC A61B 5/7282; A61B 5/7435; G01N 23/046; G01N 2223/1013; G01N 2223/108; G01N 2223/304; G01N 2223/419

(57) ABSTRACT

The nuclear medicine diagnostic apparatus according to an embodiment includes a frame apparatus and a console apparatus. The frame apparatus generates single event data based on annihilation gamma rays emitted from a subject. The console apparatus executes pairing processing to identify a combination of two pieces of the single event data. The pairing processing is executed on the single event data being stored in a first memory and corresponding to a first period of time. The console apparatus deletes the single event data that corresponds to a second period of time in the first period of time, and to which pairing processing has been applied. The console apparatus executes pairing processing on both the single event data corresponding to a third period of time in the first period of time and the single event data corresponding a fourth period of time following the first and third periods of time.

### CROSS-REFERENCE TO RELATED APPLICATIONS

This application is based upon and claims the benefit of priority from Japanese Patent Application Laid-open No. 2024-189690, filed on Oct. 29, 2024; the entire contents of which are incorporated herein by reference.

### FIELD

Embodiments disclosed herein relate generally to a nuclear medicine diagnostic apparatus.

### BACKGROUND

Conventionally, a positron emission computed tomography (PET) apparatus is known as a nuclear medicine diagnostic apparatus that can perform functional diagnosis in the biological tissue of a subject. As the resolution of the PET apparatus becomes higher, the number of arranged detectors tends to increase. In addition, PET apparatuses with a large field of view (FOV), such as a whole body PET apparatus (or a total body PET apparatus) that can scan the entire body of a subject without moving a couch, have also emerged.

Generally, in PET apparatuses, data detected by the detectors of a frame apparatus is transferred to a console apparatus to be subjected to pairing processing, but the increase in the number of detectors increases the amount of data to be transferred and subjected to the pairing processing, which in turn increases the load on data transfer and storage.

### BRIEF DESCRIPTION OF THE DRAWINGS

FIG. 1 is a diagram of an example of the configuration of a PET apparatus according to a first embodiment;

FIG. 2 is a diagram of an example of the flow of data of counting information according to the first embodiment;

FIG. 3 is a diagram of an example of a unit of pairing processing on the counting information according to the first embodiment; and

FIG. 4 is a sequence diagram of an example of a processing procedure according to the first embodiment.

### DETAILED DESCRIPTION

An embodiment of a nuclear medicine diagnostic apparatus will be described below in detail with reference to the accompanying drawings. In the following embodiment, parts denoted by the same reference signs are assumed to operate in the same way, and duplicate descriptions are omitted as appropriate.

The nuclear medicine diagnostic apparatus according to the embodiment is assumed to be a positron emission tomography (PET) apparatus for the sake of specific description. The nuclear medicine diagnostic apparatus is not limited to the PET apparatus, and may also be a PET-computed tomography (CT) apparatus. The nuclear medicine diagnostic apparatus may be a single photon emission computed tomography (SPECT) apparatus.

The nuclear medicine diagnostic apparatus according to the embodiment includes a frame apparatus and a console apparatus. The frame apparatus includes a detector, first processing circuitry, and a data acquisition system (DAS). The detector is configured to detect annihilation gamma rays emitted from a subject. The first processing circuitry is configured to generate single event data based on a detection result of the detector. The DAS is configured to transfer the single event data generated by the first processing circuitry to the console apparatus. The console apparatus includes a first memory and second processing circuitry. The first memory is configured to store the single event data transferred from the frame apparatus. The second processing circuitry is configured to execute pairing processing to identify a combination of two pieces of the single event data corresponding to two annihilation gamma rays emitted in opposite directions. The pairing processing is executed on pieces of the single event data that are stored in the first memory and correspond to a first period of time. The second processing circuitry is configured to delete, from the first memory, the single event data that corresponds to a second period of time in the first period of time, and to which the pairing processing has been applied. The second processing circuitry is configured to execute the pairing processing on both: pieces of the single event data that correspond to a third period of time in the first period of time, that are stored in the first memory, and to which the pairing processing has been applied, and pieces of the single event data that correspond to a fourth period of time following the first period of time and following the third period of time, and that is stored in the first memory.

### First Embodiment

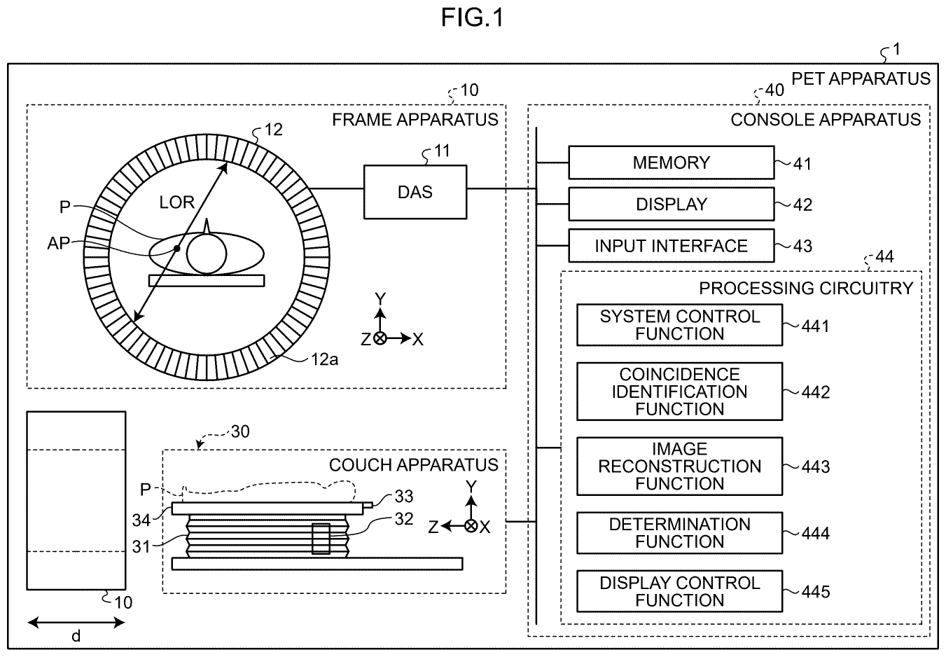

FIG. 1 is a diagram of an example of the configuration of a PET apparatus 1 according to a first embodiment. As illustrated in FIG. 1, the PET apparatus 1 includes a frame apparatus 10, a couch apparatus 30, and a console apparatus 40.

In the present embodiment, the central axis of a detector 12 or the longitudinal direction of a couchtop 33 of the couch apparatus 30 is defined as a Z-axis direction, the axial direction that is orthogonal to the Z-axis direction and is horizontal to a floor surface is defined as an X-axis direction, and the axial direction that is orthogonal to the Z-axis direction and is perpendicular to the floor surface is defined as a Y-axis direction. FIG. 1 illustrates a plurality of the frame apparatuses 10 for convenience of description, but in the actual configuration of the PET apparatus 1, there is only one frame apparatus 10.

The frame apparatus 10 includes a data acquisition system (DAS) 11 and the detector 12. The detector 12 detects gamma rays (annihilation gamma rays) emitted from within a subject P to which a drug labeled with a radioisotope that emits positrons (a positron-emitting nuclide) has been administered. In the PET apparatus 1, detecting gamma rays emitted from within the subject P is also referred to as scanning the subject P.

The detector 12 includes a plurality of detector modules 12a arranged in a ring shape surrounding the subject P. The detector 12 is an example of a collection unit that collects counting information on gamma rays emitted from the subject P. The counting information includes, for example, the detection position, the energy value, and the detection time (collection time) of gamma rays. Note that the detection time may be an absolute time or an elapsed time from the point in time of starting the scanning.

The detector module 12a scintillates and emits light in response to gamma rays emitted from within the subject P. The detector module 12a detects the emitted light and converts it into an electric signal corresponding to the energy. The detector module 12a includes, for example, a scintillator array and a silicon-photomultiplier (SiPM) array. Alternatively, the detector module 12a may include a scintillator array and a photomultiplier (PMT). Note that the detector module 12a may be referred to as the detector.

In the PET apparatus 1, the frame apparatus 10 includes the detectors 12. The detectors 12 are arranged in the X-axis direction. The detectors 12 are arranged in rows in the X-axis direction, thereby enlarging the field of view (FOV) of the PET apparatus 1 in the X-axis direction (the body axis direction of the subject P). For example, when a length d of the frame apparatus 10 in the X-axis direction is about 2 meters, the entire body of the subject P can be scanned simultaneously. The PET apparatus 1 including the frame apparatus 10 that is large enough to scan the entire body of the subject P simultaneously is called a whole body PET apparatus or a total body PET apparatus. When the PET apparatus 1 is the whole body PET apparatus, the subject P can be scanned without moving the couch apparatus 30 in the X-axis direction. Note that the PET apparatus 1 is not limited to the whole body PET apparatus, and may also scan the subject P while moving the subject P.

The DAS 11 transfers Single Data of gamma rays based on an output signal of the detector module 12a to the console apparatus 40. The DAS 11 is an example of a transfer unit.

The couch apparatus 30 is an apparatus for placing and moving the subject P to be scanned, and includes a base 31, a couch drive unit 32, the couchtop 33, and a couchtop support frame 34. The base 31 is a housing that supports the couchtop support frame 34 movably in the vertical direction. The couch drive unit 32 is a motor or actuator that moves the couchtop 33 on which the subject P is placed in the long axis direction of the couchtop 33. The couch drive unit 32 moves the couchtop 33 in accordance with control by the console apparatus 40 or control by the frame apparatus 10. The couchtop 33 provided on the upper surface of the couchtop support frame 34 is a plate on which the subject P is placed. Note that the couch drive unit 32 may move the couchtop support frame 34 in the long axis direction of the couchtop 33 in addition to the couchtop 33. When the subject P is scanned, the couch apparatus 30 may move the couchtop 33 by the step & shoot method, which alternates between scanning and moving the couchtop 33, or may move the couchtop 33 by the continuous couch movement method, which moves the couchtop 33 while scanning.

When the PET apparatus 1 is the whole body PET apparatus, the couch apparatus 30 does not need to move the couchtop 33 in the Z-axis direction because the subject P can be scanned without moving the couch apparatus 30 as described above.

The console apparatus 40 includes a memory 41, a display 42, an input interface 43, and processing circuitry 44. Note that the console apparatus 40 is described as a separate unit from the frame apparatus 10, but the frame apparatus 10 may include the console apparatus 40 or some of the components of the console apparatus 40.

The memory 41 is implemented by, for example, a random-access memory (RAM), a semiconductor memory element such as a flash memory, a hard disk, an optical disc, or the like. The memory 41 stores, for example, projection data and CT image data. For example, the memory 41 stores computer programs that cause circuits included in the PET apparatus 1 to implement various functions. The memory 41 may be implemented by a group of servers (a cloud) connected to the PET apparatus 1 via a network. The memory 41 also stores Single Data transferred from the frame apparatus 10. The memory 41 is an example of a first memory.

The display 42 displays various kinds of information. For example, the display 42 outputs medical images (PET images) generated by the processing circuitry 44, a graphical user interface (GUI) for receiving various operations from an operator, and the like. For example, as the display 42, a liquid crystal display (LCD), an organic electroluminescence display (OELD), a plasma display, or any other displays can be used as appropriate. The display 42 may be provided in the frame apparatus 10. The display 42 may be of a desktop type or may be configured as a tablet terminal or the like capable of wireless communication with the console apparatus 40 itself.

The input interface 43 receives various input operations from the operator, converts the received input operations into electric signals, and outputs them to the processing circuitry 44. For example, the input interface 43 receives from the operator collection conditions when projection data is collected, reconstruction conditions when CT images are reconstructed, image processing conditions when post-processed images are generated from CT images, and the like. As the input interface 43, for example, a mouse, a keyboard, a trackball, a switch, a button, a joystick, a touchpad, a touch panel display, and the like can be used as appropriate.

Note that in the present embodiment, the input interface 43 is not limited to those with physical operating components, such as a mouse, a keyboard, a trackball, a switch, a button, a joystick, a touchpad, and a touch panel display. For example, an electric signal processing circuit that receives electric signals corresponding to input operations from an external input device provided separately from the apparatus and outputs these electric signals to the processing circuitry 44 is also included in examples of the input interface 43. The input interface 43 is an example of an input unit. The input interface 43 may be provided in the frame apparatus 10. The input interface 43 may also be configured as a tablet terminal or the like capable of wireless communication with the console apparatus 40 itself. The input interface 43 may be provided in the frame apparatus 10. The input interface 43 may also be configured as a tablet terminal or the like capable of wireless communication with the console apparatus 40 itself.

The processing circuitry 44 controls the operation of the entire PET apparatus 1. The processing circuitry 44 includes, for example, a system control function 441, a coincidence identification function 442, an image reconstruction function 443, a determination function 444, and a display control function 445. The system control function 441 is an example of a controller. The coincidence identification function 442 is an example of a coincidence identification unit. The image reconstruction function 443 is an example of an image reconstruction unit. The determination function 444 is an example of a determination unit. The display control function 445 is an example of a display control unit. In the embodiment, each processing function performed by the system control function 441, the coincidence identification function 442, the image reconstruction function 443, the determination function 444, and the display control function 445 is stored in the memory 41 in the form of a computer program executable by a computer. The processing circuitry 44 is a processor that reads computer programs from the memory 41 and executes them to implement the functions corresponding to the respective computer programs. In other words, the processing circuitry 44 that has read the respective computer programs will have the respective functions illustrated inside the processing circuitry 44 in FIG. 1. The processing circuitry 44 is an example of second processing circuitry.

Note that in FIG. 1, a single processor is described as implementing the system control function 441, the coincidence identification function 442, the image reconstruction function 443, the determination function 444, and the display control function 445, but plural independent processors may be combined to configure the processing circuitry 44, and each processor may execute the computer program to implement the function. In FIG. 1, a single memory circuit such as the memory 41 is described as storing the computer program corresponding to each processing function, but plural memory circuits may be arranged in a distributed manner, and the processing circuitry 44 may read the corresponding computer program from an individual memory circuit.

The term “processor” used in the above description means, for example, circuits such as a central processing unit (CPU), a graphical processing unit (GPU), an application specific integrated circuit (ASIC), a programmable logic device (e.g., a simple programmable logic device (SPLD), a complex programmable logic device (CPLD), and a field programmable gate array (FPGA)). The processor reads and executes the computer programs stored in the memory 41 to perform the functions. Note that instead of storing the computer programs in the memory 41, the computer programs may be incorporated directly into the circuit of the processor. In this case, the processor reads and executes the computer programs incorporated into the circuit to implement the functions.

The system control function 441 controls the various functions of the processing circuitry 44 based on input operations received from the operator via the input interface 43. The system control function 441 performs overall control of the PET apparatus 1 by controlling the parts of the frame apparatus 10 and the console apparatus 40. For example, the system control function 441 controls the couch drive unit 32 to move the couchtop 33. The system control function 441 controls each component of the PET apparatus 1 to execute scanning. For example, the system control function 441 controls the detector 12 to collect the counting information of the annihilation gamma rays emitted from the subject P.

The coincidence identification function 442 performs pairing processing on the counting information transferred from the frame apparatus 10.

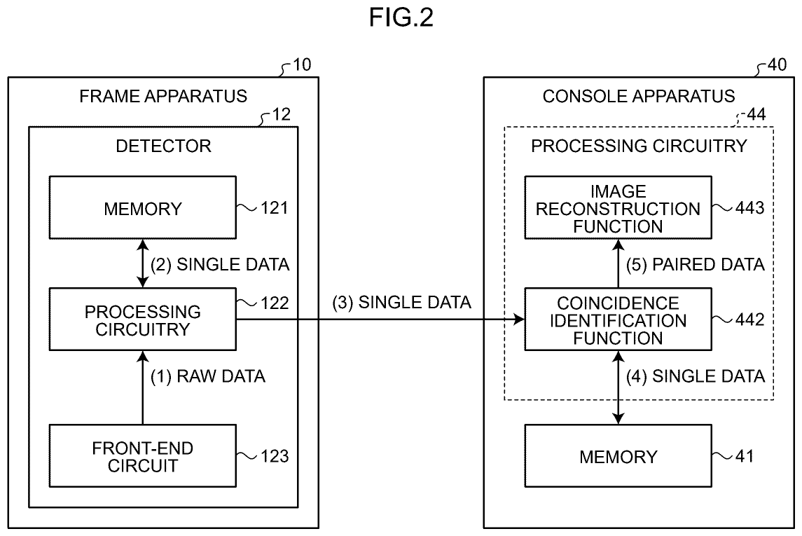

The flow of data transfer of the counting information from the frame apparatus 10 to the console apparatus 40 will be described using FIG. 2. FIG. 2 is a diagram of an example of the flow of data according to the first embodiment. Note that Raw Data, Single Data, and Paired Data illustrated in FIG. 2 are all the counting information of the annihilation gamma rays, but the state of data processing is different. The details of each data will be described along with the procedure of data transfer.

As illustrated in FIG. 2, the detector 12 includes a memory 121, processing circuitry 122, and a front-end circuit 123. Note that FIG. 2 illustrates one detector 12, but the frame apparatus 10 may include a plurality of the detectors 12.

The memory 121 is implemented by, for example, a RAM, a semiconductor memory element such as a flash memory, or the like. The memory 121 stores single event data generated by the processing circuitry 122. The memory 121 is an example of a second memory.

The processing circuitry 122 is, for example, a processor such as an FPGA. The processing circuitry 122 is an example of the generation unit. The processing circuitry 122 may also be an example of the transfer unit. The processing circuitry 122 is an example of first processing circuitry.

The front-end circuit 123 is, for example, a circuit that generates the counting information including the detection position, the energy value, and the detection time of the annihilation gamma rays based on the electric signal output from the detector module 12a. The data generated by the front-end circuit 123 is called Raw Data. The front-end circuit 123 sends out Raw Data to the processing circuitry 122. Raw Data is an example of a detection result by the detector module 12a.

The processing circuitry 122 performs arithmetic processing to correct energy information and timing information on Raw Data acquired from the front-end circuit 123, and converts Raw Data to Single Data. Single Data is also referred to as single event data or single list mode data.

The annihilation gamma rays collected by the PET apparatus 1 refer to gamma rays (photons) generated by a combination of positively charged electrons (positrons) and normally negatively charged electrons. The annihilation gamma rays are emitted in nearly-180-degree opposite directions, each having an energy of 511 keV (the rest mass energy of an electron). By simultaneously detecting two annihilation gamma rays emitted in the nearly-180-degree opposite directions by separate detector modules 12a, it can be seen that a radiation source AP (illustrated in FIG. 1) is on the straight line connecting the two detector modules 12a. This line connecting the two detector modules 12a that have detected the annihilation gamma rays is called the line of response (LOR) (illustrated in FIG. 1).

Single Data is data in which a pair of two annihilation gamma rays emitted from the subject P is not identified and the counting information pertaining to the detection of an individual annihilation gamma ray is recorded as a single event.

The processing circuitry 122 temporarily stores the generated Single Data in the memory 121. The processing circuitry 122 also transfers Single Data stored in the memory 121 to the console apparatus 40 at each specified data size or collection time. Note that Single Data is transferred from the frame apparatus 10 to the console apparatus 40 via the DAS 11, although omitted in FIG. 2.

For example, the processing circuitry 122, when Single Data stored in the memory 121 reaches the specified data size or when Single Data for a specified time length is stored in the memory 121, transfers Single Data stored in the memory 121 as one group to the console apparatus 40. The processing circuitry 122 also deletes the counting information that has already been transferred to the console apparatus 40, from the memory 121. Then, the processing circuitry 122, when Single Data stored in the memory 121 again reaches the specified data size or when the counting information for the specified time length is again stored in the memory 121, transfers Single Data stored in the memory 121 as the next one group to the console apparatus 40. Accordingly, the processing circuitry 122 temporarily stores the generated Single Data in the memory 121, and then transfers it as a lump in a certain unit to the console apparatus 40.

Note that the processing circuitry 122 may sort Single Data by the detection time included in Single Data, and then transfer it.

The specified data size and the specified time length may be predetermined, for example, in accordance with the processing speed of the processing circuitry 44 of the console apparatus 40 and the sizes of the memories 41 and 121 of the detector 12 and the console apparatus. For example, the specified data size and the specified time length are determined such that when Single Data is grouped in units of the specified data size or the specified time length, one group is small enough to be processed by the console apparatus 40 and the detector 12. Whether the division unit of Single Data is the specified data size or the specified time length may be predetermined, or may be dynamically determined by the determination function 444 of the console apparatus 40.

Note that the processing circuitry 122 does not have to always temporarily store Single Data in the memory 121, and may always temporarily store Single Data in the memory 121 only when that temporary storage is determined to be necessary by the determination function 444 of the console apparatus 40. Note that the necessity or unnecessity of temporary storage may be determined by the processing circuitry 122.

Note that if determining that the temporary storage of Single Data is unnecessary, the processing circuitry 122 does not temporarily store Single Data in the memory 121 or divide Single Data into plural groups. In this case, the processing circuitry 122 transfers Single Data to the console apparatus 40 each time it is collected. Note that even if Single Data is not stored in the memory 121, Single Data may be stored in a buffer memory or the like for transfer processing.

When Single Data is transferred from the frame apparatus 10 to the console apparatus 40, the processing circuitry 44 of the console apparatus 40 performs the pairing processing on Single Data.

More precisely, the coincidence identification function 442 of the processing circuitry 44 temporarily stores Single Data transferred from the frame apparatus 10 in the memory 41. Then, the coincidence identification function 442 generates Paired Data by performing the pairing processing on the temporarily stored Single Data. Note that when Single Data is not sorted in the frame apparatus 10, the coincidence identification function 442 sorts Single Data by the detection time included in Single Data before the pairing processing.

The pairing processing is the processing of identifying a combination of two Single Data corresponding to the two annihilation gamma rays emitted in opposite directions out of Single Data transferred from the frame apparatus 10. For example, the coincidence identification function 442 pairs each event detected within a specified timing window in order to identify two events detected at approximately the same time. Note that the timing window is also referred to as a time window or a time width. In other words, the coincidence identification function 442 generates Paired Data with the combination of two Single Data the collection times of which are within a timing window width of a certain time out of Single Data transferred from the frame apparatus 10 as information meaning that the two annihilation gamma rays have coincidently been counted.

Paired Data is data in which two Single Data corresponding to the two annihilation gamma rays are associated with each other as a pair. Paired Data is an example of coincidence information.

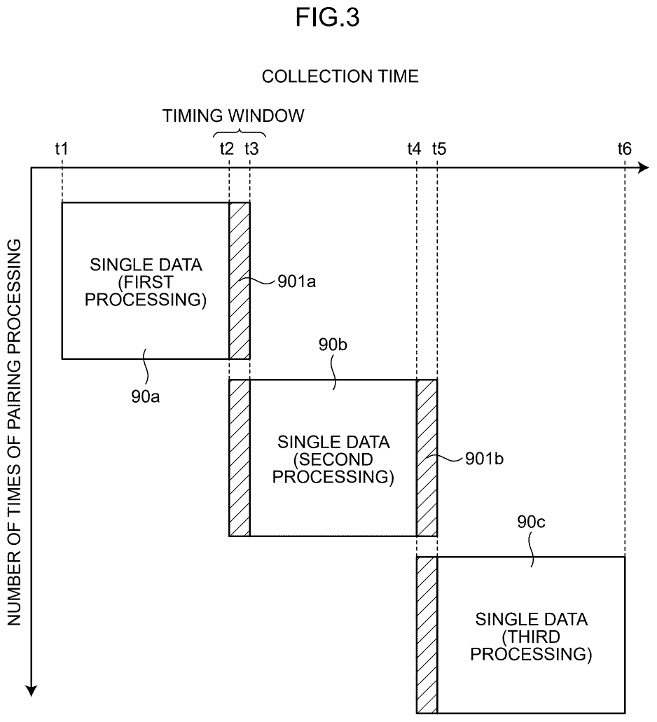

The coincidence identification function 442 performs the pairing processing on Single Data transferred while being divided into groups sequentially for each group. When the pairing processing on Single Data of one group is completed, the coincidence identification function 442 deletes the processed Single Data from the memory 41 except for a range necessary for the pairing processing on Single Data of the next group. The range necessary for the pairing processing on Single Data of the next group is an example of a specified non-deletion range. The pairing processing and the deletion of Single Data will be described later using FIG. 3.

When Single Data is transferred each time it is collected, rather than being transferred while being divided into the groups, the coincidence identification function 442 stores all Single Data collected in one scan in the memory 41. After all Single Data collected in one scan are stored in the memory 41, the coincidence identification function 442 performs the pairing processing on Single Data of the one scan.

The image reconstruction function 443 generates a PET image by reconstructing Paired Data generated by the coincidence identification function 442. For example, the image reconstruction function 443 executes reconstruction by the maximum likelihood-expectation maximization (ML-EM) method or the ordered subset-expectation maximization (OS-EM) method, which is a faster version of the ML-EM method. Note that the image reconstruction function 443 may be implemented by a processor (a GPU or the like) different from the coincidence identification function 442.

Referring back to FIG. 1, the determination function 444 determines whether to temporarily store the collected Single Data in the memory 121 based on the count rate of the annihilation gamma rays or drug information about a drug administered to the subject P. The determination whether to temporarily store the collected Single Data in the memory 121 is, in other words, a determination about whether to transfer the collected Single Data to the console apparatus 40 while being divided in group units or to transfer them each time they are collected without grouping them.

For example, the determination function 444 may calculate the count rate, which indicates the number of annihilation gamma rays detected per unit time during scanning, and determine that the temporary storage of Single Data is necessary when the calculated count rate is a threshold or more.

The height of the count rate of the annihilation gamma rays can be estimated based on the drug information about the drug administered to the subject P. Therefore, the determination function 444 may determine that the temporary storage of Single Data is necessary when the count rate of the annihilation gamma rays is estimated to be the threshold or more based on the type of the drug administered to the subject P. The type of the drug administered to the subject P is included, for example, in an examination order transmitted from the Radiology Information System (RIS) to the PET apparatus 1 via a network. Note that the type of the drug administered to the subject P may be entered by a user's operation via the input interface 43.

The determination function 444 may also determine which of the specified data size and the specified time length is to be used as the division unit of Single Data based on the count rate of the annihilation gamma rays or the drug information about the drug administered to the subject P.

The display control function 445 controls the display 42 to display various screens. For example, the display control function 445 displays a GUI that can receive an operation to start scanning and various other operations by the user on the display 42. The display control function 445 may also display the PET image generated by the image reconstruction function 443 on the display 42.

The pairing processing on Single Data transferred while being combined into groups and the deletion of Single Data will be described in more detail using FIG. 3.

FIG. 3 is a diagram of an example of a unit of the pairing processing of the counting information (Single Data) according to the first embodiment. In the example illustrated in FIG. 3, Single Data collected by one scan is divided into three groups, but the number of such groups is an example and is not limited to this example.

The horizontal axis of FIG. 3 indicates the length of the collection time, and the vertical axis indicates the number of times of the pairing processing. The coincidence identification function 442, each time the pairing processing of one group is performed, deletes Single Data of the group from the memory 41, but Single Data contained in the time length equivalent to the timing window at the end of each group may still be valid for the pairing processing on the next group, and therefore, is not subject to deletion. The time length between t2 and t3 and the time length between t4 and t5 illustrated in FIG. 3 are the same time length as the specified timing window in the pairing processing. Note that the time length of the timing window is generally a very short time in nanoseconds or picoseconds.

For example, assume that Single Data 90a of the first group transferred from the frame apparatus 10 is the counting information with a collection time of t1 to t3. Out of Single Data 90a of the first group, Single Data in the range corresponding to the timing window width from the last of the collection time, namely, Single Data with a collection time of t2 to t3, is a non-deletion range 901a of the first group. The period of time from the collection time t1 to a processing time t3 is an example of the first period of time. The period of time from the collection time t1 to a processing time t2 is an example of the second period of time. The period of time from the collection time t2 to a processing time t3 is an example of the third period of time. The period of time from the collection time t3 to a processing time t5 is an example of the fourth period of time.

The coincidence identification function 442 performs the pairing processing to identify a combination of two Single Data corresponding to the two annihilation gamma rays emitted in opposite directions out of Single Data 90a of the first group transferred from the frame apparatus 10. After performing the pairing processing on Single Data 90a of the first group, the coincidence identification function 442 deletes Single Data other than Single Data with the collection time of t2 to t3 out of Single Data 90a of the first group, from the memory 41.

The reason why Single Data with the collection time of t2 to t3 is not deleted is that, the two annihilation gamma rays to be paired may be transferred separately as Single Data 90a of the first group and Single Data 90b of the next second group. In such a case, if all Single Data 90a of the first group is deleted before the pairing processing on Single Data 90b of the second group, the annihilation gamma rays to be paired with the annihilation gamma rays contained in Single Data 90b of the second group may be deleted. Therefore, the coincidence identification function 442 does not delete the range corresponding to the timing window width from the last collection time out of Single Data 90a of the first group until the pairing processing on Single Data 90b of the second group is completed.

Then, the coincidence identification function 442 performs the pairing processing on Single Data contained in the non-deletion range 901a of the first group and Single Data 90b of the second group transferred next from the frame apparatus 10. After performing the pairing processing on Single Data contained in the non-deletion range 901a of the first group and Single Data 90b of the second group, the coincidence identification function 442 deletes Single Data other than Single Data with the collection time of t4 to t5 out of Single Data 90b of the second group. At this time, the coincidence identification function 442 also deletes Single Data contained in the non-deletion range 901a of the first group.

The reason why the coincidence identification function 442 does not delete Single Data with the collection time of t4 to t5 (that is, Single Data contained in a non-deletion range 901b of the second group) out of Single Data 90b of the second group, but leaves it is that, it is used for the pairing processing on Single Data 90c of a third group.

Then, the coincidence identification function 442 performs the pairing processing on Single Data contained in the non-deletion range 901b of the second group and Single Data 90c of the third group transferred next from the frame apparatus 10. In the example illustrated in FIG. 3, Single Data 90c of the third group is the last data transferred from the frame apparatus 10, and thus the coincidence identification function 442 deletes Single Data contained in the non-deletion range 901b of the second group, and after performing the pairing processing on Single Data 90c of the third group, deletes all Single Data from the memory 41.

The following describes the procedure of processing executed by the PET apparatus 1 configured as described above.

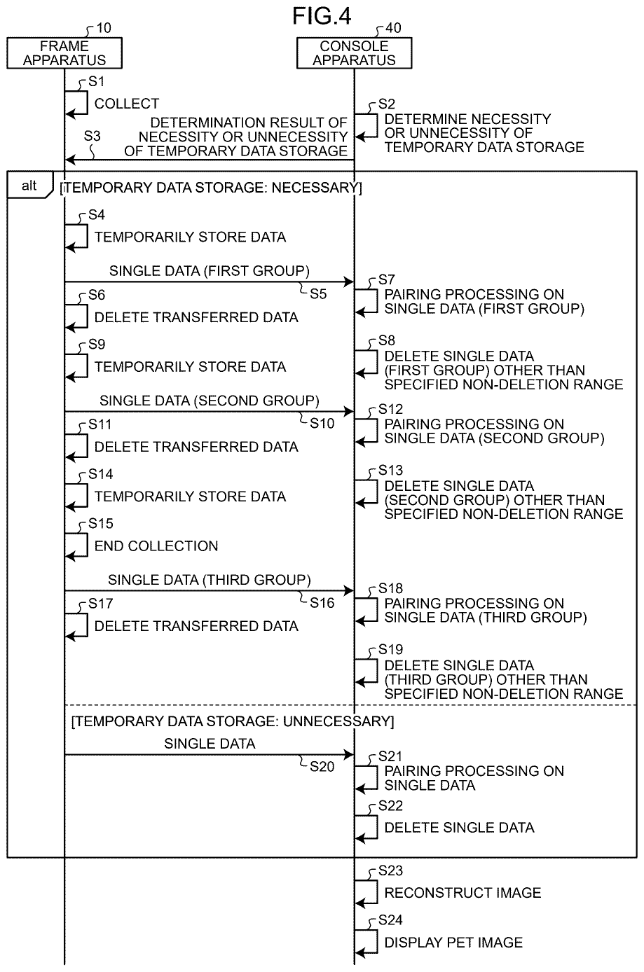

FIG. 4 is a sequence diagram of an example of a processing procedure according to the first embodiment. The processing according to this sequence diagram starts, for example, when the operation to start scanning is input by the user.

First, the frame apparatus 10 starts detection of the annihilation gamma rays emitted from within the subject P by the detector 12 and collection of the counting information of the detected annihilation gamma rays (S1). For example, the front-end circuit 123 of each detector 12 generates Raw Data of the counting information based on the electric signal output from the detector module 12a. The processing circuitry 122 generates Single Data from Raw Data generated by front-end circuit 123.

The determination function 444 of the console apparatus 40 determines the necessity or unnecessity of the temporary storage of Single Data in the memory 121 based on, for example, the drug information about the drug administered to the subject P or the estimated count rate of Single Data (S2).

Then, the determination function 444 of the console apparatus 40 transmits a determination result of the necessity or unnecessity of the temporary storage of Single Data to the processing circuitry 122 of the frame apparatus 10 (S3).

The processing circuitry 122 stores the collected Single Data in the memory 121 in accordance with the determination result by the determination function 444 of the console apparatus 40. Specifically, in response to determining that the temporary storage of Single Data is necessary (alt “TEMPORARY DATA STORAGE: NECESSARY”), the processing circuitry 122 of the frame apparatus 10 temporarily stores the generated Single Data in the memory 121 (S4).

Then, the processing circuitry 122 of the frame apparatus 10, when Single Data stored in the memory 121 reaches the specified data size or when Single Data for the specified time length is stored in the memory 121, transfers Single Data stored in the memory 121 as Single Data 90a of the first group to the console apparatus 40 via the DAS 11 (S5). The coincidence identification function 442 of the processing circuitry 44 of the console apparatus 40 temporarily stores Single Data 90a of the first group transferred from the frame apparatus 10 in the memory 41.

The processing circuitry 122 of the frame apparatus 10 deletes Single Data 90a of the first group, which has already been transferred to the console apparatus 40, from the memory 121 (S6).

Then, the coincidence identification function 442 of the processing circuitry 44 of the console apparatus 40 generates Paired Data by sorting the temporarily stored Single Data 90a of the first group by the detection time and then executing the pairing processing (S7). The coincidence identification function 442 stores the generated Paired Data in the memory 41.

Then, the coincidence identification function 442 of the processing circuitry 44 of the console apparatus 40 deletes Single Data 90a of the first group other than the specified non-deletion range 901a, from the memory 41 (S8).

Also after transferring Single Data 90a of the first group at S5, the frame apparatus 10 continues to temporarily store the newly collected counting information in the memory 121 (S9).

The processing circuitry 122 of the frame apparatus 10, when Single Data stored in the memory 121 again reaches the specified data size or when Single Data for the specified time length is again stored in the memory 121, transfers Single Data stored in the memory 121 as Single Data 90b of the second group to the console apparatus 40 via the DAS 11 (S10). The coincidence identification function 442 of the processing circuitry 44 of the console apparatus 40 temporarily stores Single Data 90b of the second group transferred from the frame apparatus 10 in the memory 41.

The processing circuitry 122 of the frame apparatus 10 deletes Single Data 90b of the second group, which has already been transferred to the console apparatus 40, from the memory 121 (S11).

Then, the coincidence identification function 442 of the processing circuitry 44 of the console apparatus 40 generates Paired Data by sorting Single Data of the specified non-deletion range 901a out of Single Data 90a of the first group and the temporarily stored Single Data 90b of the second group by the detection time and then performing the pairing processing (S12). The coincidence identification function 442 stores the generated Paired Data in the memory 41.

Then, the coincidence identification function 442 of the processing circuitry 44 of the console apparatus 40 deletes Single Data 90b of the second group other than the specified non-deletion range 901b, from the memory 41 (S13). At this time, the coincidence identification function 442 also deletes Single Data of the specified non-deletion range 901a, which was left without being deleted at S8, from the memory 41.

Also after transferring Single Data 90b of the second group at S10, the frame apparatus 10 continues to temporarily store the newly collected counting information in the memory 121 (S14).

Here, the scanning of the subject P ends, and the collection of the counting information ends (S15). The processing circuitry 122 of the frame apparatus 10 transfers Single Data stored in the memory 121 as Single Data 90c of the third group to the console apparatus 40 via the DAS 11 (S16). The coincidence identification function 442 of the processing circuitry 44 of the console apparatus 40 temporarily stores Single Data 90c of the third group transferred from the frame apparatus 10 in the memory 41.

The processing circuitry 122 of the frame apparatus 10 deletes Single Data 90c of the third group, which has already been transferred to the console apparatus 40, from the memory 121 (S17).

Then, the coincidence identification function 442 of the processing circuitry 44 of the console apparatus 40 generates Paired Data by sorting Single Data of the specified non-deletion range 901b out of Single Data 90b of the second group and the temporarily stored Single Data 90c of the third group by the detection time and then performing the pairing processing (S18). The coincidence identification function 442 stores the generated Paired Data in the memory 41.

Here, since the pairing processing on all Single Data has ended, the coincidence identification function 442 of the processing circuitry 44 of the console apparatus 40 deletes Single Data 90c of the third group from the memory 41 (S19). At this time, the coincidence identification function 442 also deletes Single Data of the specified non-deletion range 901b, which was left without being deleted at S13, from the memory 41.

If the temporary storage of Single Data is determined to be unnecessary at the S2 processing (alt “TEMPORARY DATA STORAGE: UNNECESSARY”), the processing circuitry 122 of the frame apparatus 10 does not accumulate the collected Single Data, but transfers Single Data to the console apparatus 40 via the DAS 11 each time it is collected (S20). The transfer of Single Data continues until the scanning of the subject P ends and the collection of the counting information ends. The coincidence identification function 442 of the processing circuitry 44 of the console apparatus 40 temporarily stores Single Data transferred from the frame apparatus 10 in the memory 41.

After all Single Data collected in one scan are stored in the memory 41, the coincidence identification function 442 of the processing circuitry 44 of the console apparatus 40 sorts Single Data for that one scan by the detection time, and then performs the pairing processing (S21). The coincidence identification function 442 stores the generated Paired Data in the memory 41.

The coincidence identification function 442 deletes Single Data from the memory 41 after the end of the pairing processing (S22).

Then, the image reconstruction function 443 of the processing circuitry 44 of the console apparatus 40 generates a PET image by reconstructing Paired Data generated by the coincidence identification function 442 and stored in the memory 41 (S23).

The display control function 445 of the processing circuitry 44 of the console apparatus 40, for example, displays the PET image generated by the image reconstruction function 443 on the display 42 (S24). Here, the processing of this sequence diagram ends.

Note that, in FIG. 4, Single Data collected in one scan is transferred while being divided into three groups by the frame apparatus 10, but the number of groups of the transferred Single Data is not limited to this example.

As described above, the PET apparatus 1 of the present embodiment includes the frame apparatus 10 and the console apparatus 40. The frame apparatus 10 collects Single Data of the annihilation gamma rays emitted from the subject P, and transfers the collected counting information to the console apparatus 40. After performing the pairing processing on Single Data 90a of the first group transferred from the frame apparatus 10, the console apparatus 40 leaves Single Data of the specified non-deletion range 901a to be used for the pairing processing on Single Data 90b of the second group transferred next from the frame apparatus 10, and deletes Single Data 90a of the first group other than the specified non-deletion range 901a. Therefore, with the PET apparatus 1 of the present embodiment, the load related to data transfer and storage can be reduced compared to the case where Single Data collected in one scan is transferred simultaneously from the frame apparatus 10 to the console apparatus 40.

For example, in a configuration with a large number of arranged detectors 12, such as a whole body PET apparatus, or in a configuration with a large number of detector modules 12a included in one detector 12 due to higher resolution, a large amount of Single Data is collected in one scan. In the PET apparatus 1 with such a configuration, when Single Data collected in one scan is transferred to the console apparatus 40 at a time, the data transfer load becomes high. Even if Single Data is transferred little by little from the frame apparatus 10 to the console apparatus 40, if the pairing processing is performed after all Single Data collected in one scan are accumulated on the console apparatus 40, the amount of data stored on the console apparatus 40 will increase.

In contrast, when Single Data for which the pairing processing has ended is deleted first, as in the PET apparatus 1 of the present embodiment, the amount of Single Data stored in the memory 41 can be reduced. This can reduce the memory capacity of the memory 121 on the frame apparatus 10 and the memory 41 on the console apparatus 40 to be low even when the amount of Single Data collected in one scan increases.

The PET apparatus 1 of the present embodiment does not simply delete Single Data belonging to a group for which the pairing processing has ended, but leaves the range that may contain Single Data that will be paired with Single Data belonging to a group to be transferred next from the frame apparatus 10 without deleting it, thereby reducing the number of omissions of the pairing of Single Data.

The frame apparatus 10 of the PET apparatus 1 of the present embodiment also includes the memory 121 that stores the collected Single Data. The frame apparatus 10 of the PET apparatus 1 of the present embodiment transfers Single Data stored in the memory 121 to the console apparatus 40 at each specified data size or collection time. Therefore, with the PET apparatus 1 of the present embodiment, Single Data can be transferred to the console apparatus 40 while being divided in an appropriate size.

The frame apparatus 10 of the PET apparatus 1 of the present embodiment, when Single Data stored in the memory 121 reaches the specified data size or when Single Data for the specified time length is stored in the memory 121, transfers Single Data stored in the memory 121 as Single Data 90a of the first group to the console apparatus 40. The frame apparatus 10 of the PET apparatus 1 of the present embodiment deletes Single Data 90a of the first group from the memory 121 after transferring Single Data 90a of the first group. Therefore, with the PET apparatus 1 of the present embodiment, even when a large amount of Single Data is collected, data can be avoided from overflowing from the memory 121 of the frame apparatus 10.

The frame apparatus 10 of the PET apparatus 1 of the present embodiment, when Single Data collected and stored in the memory 121 after Single Data 90a of the first group again reaches the specified data size or when Single Data for the specified time length is again stored in the memory 121, transfers Single Data stored in the memory 121 as Single Data 90b of the second group to the console apparatus 40, and after the transfer, deletes Single Data 90b of the second from the memory 121. Accordingly, with the PET apparatus 1 of the present embodiment, the frame apparatus 10 sequentially transfers the collected Single Data to the console apparatus 40 at each specified data size or specified time length, and thus the console apparatus 40 can perform the pairing processing sequentially starting with Single Data of the group transferred first. Single Data that has already been transferred is deleted from the memory 121 of the frame apparatus 10, and thus the amount of data stored in the memory 121 of the frame apparatus 10 can be reduced even when a large amount of Single Data is collected.

After the pairing processing on Single Data 90a of the first group, the console apparatus 40 of the PET apparatus 1 of the present embodiment deletes Single Data other than the specified non-deletion range 901a out of Single Data 90a of the first group, from the memory 41. The console apparatus 40 of the PET apparatus 1 of the present embodiment performs the pairing processing on Single Data included in the specified non-deletion range 901a out of Single Data 90a of the first group and Single Data 90b of the second group. Therefore, with the PET apparatus 1 of the present embodiment, Single Data for which the pairing processing has ended is deleted to free up space in the memory 41, and even when two Single Data to be paired are transferred across two groups, the pair can be identified.

The console apparatus 40 of the PET apparatus 1 of the present embodiment generates Paired Data with the combination of two Single Data the collection times of which are within a timing window width of a certain time out of Single Data 90a of the first group as information meaning that the two annihilation gamma rays have coincidently been counted. The specified non-deletion range 901a used for deleting Single Data from the memory 41 is the range of the timing window width from the last of the collection time out of Single Data 90a of the first group. Therefore, with the PET apparatus 1 of the present embodiment, even when two Single Data contained within a specified timing window are transferred across two groups in order to identify two events detected almost simultaneously, the pair can be identified.

The console apparatus 40 of the PET apparatus 1 of the present embodiment determines whether to store the collected Single Data in the memory 121 of the frame apparatus 10 based on the count rate of the annihilation gamma rays or the drug information about the drug administered to the subject P. The frame apparatus 10 of the PET apparatus 1 of the present embodiment stores the collected Single Data in the memory 121 in accordance with the determination result by the console apparatus 40. For example, when the amount of Single Data collected in one scan is large, it is effective to reduce the load related to data transfer and storage by transferring Single Data while being divided in certain units, but when the amount of Single Data collected in one scan is small, there is no problem in transferring Single Data from the frame apparatus 10 to the console apparatus 40 as soon as it is collected without performing such processing. Therefore, with the PET apparatus 1 of the present embodiment, an appropriate transfer method can be adopted in accordance with the amount of Single Data collected in one scan.

### Second Embodiment

In the first embodiment described above, the console apparatus 40 of the PET apparatus 1 identifies the specified non-deletion ranges 901a and 901b out of Single Data transferred from the frame apparatus 10 for each group, and performs the pairing processing for each group while deleting the other Single Data with the specified non-deletion ranges 901a and 901b left. In this second embodiment, Single Data corresponding to the specified non-deletion ranges 901a and 901b is identified on the frame apparatus 10, and Single Data corresponding to the specified non-deletion ranges 901a and 901b that have already been transferred is transferred in a duplicate manner together with Single Data of the next group.

The PET apparatus 1 of the present embodiment includes the frame apparatus 10, the couch apparatus 30, and the console apparatus 40, as in the first embodiment described in FIG. 1.

The detector 12 of the frame apparatus 10 of the present embodiment includes the memory 121, the processing circuitry 122, and the front-end circuit 123, as in the first embodiment described in FIG. 2. The flow of Raw Data, Single Data, and Paired Data is also similar to that illustrated in FIG. 2.

In the first embodiment, the processing circuitry 122 of the frame apparatus 10 transfers Single Data to the console apparatus 40, and then deletes Single Data that has already been transferred, from the memory 121. In contrast, the processing circuitry 122 of the frame apparatus 10 of the present embodiment does not delete from the memory 121 Single Data of the specified non-deletion range to be used for the pairing processing on Single Data of the group to be transferred next out of Single Data that has already been transferred. The processing circuitry 122 of the frame apparatus 10 transfers Single Data of the specified non-deletion range out of Single Data of the previously transferred group to the console apparatus 40 together with Single Data of the group to be transferred next.

In more detail, the processing circuitry 122 of the frame apparatus 10 of the present embodiment, when Single Data stored in the memory 121 reaches the specified data size or Single Data for the specified time length is stored in the memory 121, transfers Single Data stored in the memory 121 as Single Data 90a of the first group to the console apparatus 40.

After transferring Single Data 90a of the first group, the processing circuitry 122 of the frame apparatus 10 of the present embodiment deletes Single Data other than the specified non-deletion range 901a out of Single Data 90a of the first group, from the memory 121.

The specified non-deletion range 901a is, for example, the range of the timing window width from the last of the collection time out of Single Data 90a of the first group, as in the first embodiment.

Then, the processing circuitry 122 of the frame apparatus 10 of the present embodiment, when Single Data collected and stored in the memory 121 after Single Data 90a of the first group again reaches the specified data size or when Single Data for the specified time length is again stored in the memory 121, transfers Single Data 90b of the second group stored in the memory 121 and Single Data of the specified non-deletion range 901a remaining in the memory 121 without being deleted to the console apparatus 40.

Note that after transferring Single Data 90b of the second group, the processing circuitry 122 deletes the specified non-deletion range 901a of Single Data 90a of the first group, from the memory 121. After transferring Single Data 90b of the second group, the processing circuitry 122 deletes Single Data other than the specified non-deletion range 901b out of Single Data 90b of the second group, from the memory 121. Note that the deletion of Single Data 90a of the first group that has already been transferred from the memory 121 may be performed by the DAS 11. In other words, the DAS 11, after transferring pieces of Single Data 90a corresponding to the first period of time to the console apparatus 40, may delete pieces of Single Data 90a corresponding to the second period of time (a collection time of t1 to t2), which is part of the first period of time (a collection time of t1 to t2), from the memory 121.

Then, the processing circuitry 122 of the frame apparatus 10 of the present embodiment, when Single Data collected and stored in the memory 121 after Single Data 90b of the second group again reaches the specified data size or when Single Data for the specified time length is again stored in the memory 121, transfers Single Data 90c of the third group stored in the memory 121 and Single Data of the specified non-deletion range 901b remaining in the memory 121 without being deleted to the console apparatus 40. In other words, the DAS 11, when Single Data 90a of an amount corresponding to the fourth period of time (a collection time of t3 to t5) is stored in the memory 121, may transfer pieces of Single Data 90a corresponding to the third period of time (a collection time of t2 to t3) and pieces of Single Data 90a corresponding to the fourth period of time stored in the memory 121 to the console apparatus.

In the present embodiment, the coincidence identification function 442 of the processing circuitry 44 of the console apparatus 40 may delete all Single Data of the target group after the pairing processing for each group without identifying the specified non-deletion ranges 901a and 901b.

As described above, the frame apparatus 10 of the PET apparatus 1 of the present embodiment transfers the range to be used for the pairing processing on Single Data of the group to be transferred next out of Single Data that has already been transferred to the console apparatus 40 to the console apparatus 40 again together with the next group. Therefore, with the frame apparatus 10 of the PET apparatus 1 of the present embodiment, in addition to the same effect as the first embodiment, the console apparatus 40 does not need to perform the control processing to identify and to delete or not to delete the specified non-deletion ranges 901a and 901b.

### First Modification

Note that in the first and second embodiments described above, the memory 121, the processing circuitry 122, and the front-end circuit 123 are provided for each detector 12, but one memory 121 and one piece of processing circuitry 122 may be provided for the entire frame apparatus 10. In this case, the DAS 11 may include the memory 121 and the processing circuitry 122.

Alternatively, the frame apparatus 10 may include both the processing circuitry 122 provided for each detector 12 and one piece of processing circuitry (such as an FPGA) provided for the entire frame apparatus 10. In this case, the functions described as the functions of the processing circuitry 122 in each of the embodiments described above may be shared in a divided manner by the processing circuitry 122 provided for each detector 12 and one piece of processing circuitry provided for the entire frame apparatus 10.

### Second Modification

All or some of what are illustrated as the determination function 444 of the processing circuitry 44 of the console apparatus 40 in the first embodiment described above may be served by the processing circuitry 122 or the DAS 11 of the frame apparatus 10. In FIG. 4, the determination function 444 of the console apparatus 40 determines the necessity or unnecessity of the temporary storage of Single Data in the memory 121 before the first transfer, but the timing of the determination is not limited to this example, and may be during the collection of Single Data.

For example, control may be provided whereby the frame apparatus 10 transfers data to the console apparatus 40 before the memory 121 overflows when there is a sudden increase in the count rate during scanning.

More specifically, the processing circuitry 122 of the frame apparatus 10 may transfer Single Data stored in the memory 121 to the console apparatus 40 and delete it from the memory 121 when the count rate of Single Data becomes a threshold or more during the collection of Single Data. The DAS 11 may also store Single Data generated by the processing circuitry 122 in the memory 121 when the count rate of the annihilation gamma rays is greater than a preset value. The DAS 11 may transfer Single Data generated by the processing circuitry 122 to the console apparatus without storing it in the memory 121 when the count rate of the annihilation gamma rays is smaller than the preset value.

By thus changing the transfer unit of Single Data to an appropriate size during scanning, in addition to the effects of the first and second embodiments described above, it is possible to cope with high load conditions caused by the collection of a large amount of Single Data.

### Third Modification

Depending on the configuration of the PET apparatus 1, it may be assumed that Single Data to be collected is always large in amount. For this reason, the PET apparatus 1 may always temporarily store Single Data in the memory 121 of the frame apparatus 10. In this case, the PET apparatus 1 does not need to include the determination function 444 because the determination processing for the necessity or unnecessity of the temporary storage is unnecessary.

### Fourth Modification

In the first and second embodiments described above, the mode of data transfer is instructed from the console apparatus 40 to the frame apparatus 10 before the start of the transfer of Single Data, and Single Data is transferred from the frame apparatus 10 to the console apparatus 40 by a push method during the transfer. However, the data transfer method is not limited to such a procedure.

For example, a pull-type transfer method may be employed, in which the console apparatus 40 requests the transfer of Single Data to the frame apparatus 10.

Specifically, the coincidence identification function 442 of the console apparatus 40 may request the transfer of Single Data of the next group to the frame apparatus 10 when the pairing processing on the transferred Single Data ends. In the case of the pull-type transfer method, data transfer can be performed from the frame apparatus 10 at a timing according to the processing speed of the console apparatus 40.

### Fifth Modification

In the first and second embodiments described above, the specified non-deletion ranges 901a and 901b to be left for the pairing processing on the next group are the range of the timing window width from the last of the collection time of Single Data of each group, but the specified non-deletion ranges 901a and 901b are not limited to this example.

For example, the specified non-deletion ranges 901a and 901b may be longer time lengths than the timing window in order to calculate the contingent coincidence by the delayed coincidence method and to absorb deviations of a data transmission time between the detectors 12.

Note that various data handled in the present specification are typically digital data.

At least one of the embodiments described above can reduce the load related to data transfer and storage in the nuclear medicine diagnostic apparatus.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A nuclear medicine diagnostic apparatus comprising:
a frame apparatus; and
a console apparatus,
the frame apparatus including:
a detector configured to detect annihilation gamma rays emitted from a subject;
first processing circuitry configured to generate single event data based on a detection result of the detector; and
a data acquisition system (DAS) configured to transfer the single event data generated by the first processing circuitry to the console apparatus, and
the console apparatus including:
a first memory configured to store the single event data transferred from the frame apparatus; and
second processing circuitry configured to
execute pairing processing to identify a combination of two pieces of the single event data corresponding to two annihilation gamma rays emitted in opposite directions, the pairing processing being executed on pieces of the single event data that are stored in the first memory and correspond to a first period of time,
delete, from the first memory, the single event data that corresponds to a second period of time in the first period of time, and to which the pairing processing has been applied, and
execute the pairing processing on both
pieces of the single event data that correspond to a third period of time in the first period of time, that are stored in the first memory, and to which the pairing processing has been applied, and
pieces of the single event data that correspond to a fourth period of time following the first period of time and following the third period of time, and that is stored in the first memory.

2. The nuclear medicine diagnostic apparatus according to claim 1, wherein
the frame apparatus includes a second memory configured to store the single event data generated by the first processing circuitry, and
the DAS is further configured to,
when the single event data in an amount corresponding to the first period of time is stored in the second memory, transfer pieces of the single event data corresponding to the first period of time to the console apparatus, and,
when the single event data in an amount corresponding to the fourth period of time is stored in the second memory, transfer pieces of the single event data corresponding to the fourth period of time to the console apparatus.

3. The nuclear medicine diagnostic apparatus according to claim 2, wherein the DAS is further configured to delete the pieces of single event data corresponding to the first period of time from the second memory after transferring the same to the console apparatus.

4. The nuclear medicine diagnostic apparatus according to claim 2, wherein the DAS is further configured to
delete pieces of the single event data corresponding to the second period of time from the second memory after transferring the pieces of single event data corresponding to the first period of time to the console apparatus, and,
when the single event data in an amount corresponding to the fourth period of time is stored in the second memory, transfer, to the console apparatus, pieces of single event data corresponding to the third period of time and the pieces of single event data corresponding to the fourth period of time stored in the second memory.

5. The nuclear medicine diagnostic apparatus according to claim 2, wherein the DAS is further configured to store, in the second memory, the single event data generated by the first processing circuitry when a count rate of the annihilation gamma rays is greater than a preset value.

6. The nuclear medicine diagnostic apparatus according to claim 5, wherein the DAS transfers the single event data generated by the first processing circuitry to the console apparatus without storing the single event data in the second memory when the count rate of the annihilation gamma rays is smaller than the preset value.

7. The nuclear medicine diagnostic apparatus according to claim 1, wherein
the second processing circuitry is further configured to, in the pairing processing, generate, as coincidence information, a combination of two pieces of the single event data that are present within a width of a preset timing window, the coincidence information being information obtained by coincidently counting the two annihilation gamma rays, and
the third period of time has a length corresponding to the width of the preset timing window.
